SEQUENCE LISTING (11 sequences)

SEQ ID NO: 1 - protein, 1576 aa, Homo sapiens
GCGAGGCAGCCAGCGAGGGAGAGAGCGAGCGGGCGAGCCGGAGCGAGGAAGGGAAAGCGCAAGAGAGAGCGCACACGCACACACCCGCCGCGCGCACTCGCGCCCGGACCCGCACGGGGACAGCTCGGAAGTCATCAGTTCCATGGGCGAGATGCTGCTGCTGGCGAGATGTCTGCTGCTAGTCCTCGTCTCCTCGCTGCTGGTATGCTCGGGACTGGCGTGCGGACCGGGCAGGGGGTTCGGGAAGAGGAGGCACCCCAAAAAGCTGACCCCTTTAGCCTACAAGCAGTTTATCCCCAATGTGGCCGAGAAGACCCTAGGCGCCAGCGGAAGGTATGAAGGGAAGATCTCCAGAAACTCCGAGCGATTTAAGGAACTCACCCCCAATTACAACCCCGACATCATATTTAAGGATGAAGAAAACACCGGAGCGGACAGGCTGATGACTCAGAGGTGTAAGGACAAGTTGAACGCTTTGGCCATCTCGGTGATGAACCAGTGGCCAGGAGTGAAACTGCGGGTGACCGAGGGCTGGGACGAAGATGGCCACCACTCAGAGGAGTCTCTGCACTACGAGGGCCGCGCAGTGGACATCACCACGTCTGACCGCGACCGCAGCAAGTACGGCATGCTGGCCCGCCTGGCGGTGGAGGCCGGCTTCGACTGGGTGTACTACGAGTCCAAGGCACATATCCACTGCTCGGTGAAAGCAGAGAACTCGGTGGCGGCCAAATCGGGAGGCTGCTTCCCGGGCTCGGCCACGGTGCACCTGGAGCAGGGCGGCACCAAGCTGGTGAAGGACCTGAGCCCCGGGGACCGCGTGCTGGCGGCGGACGACCAGGGCCGGCTGCTCTACAGCGACTTCCTCACTTTCCTGGACCGCGACGACGGCGCCAAGAAGGTCTTCTACGTGATCGAGACGCGGGAGCCGCGCGAGCGCCTGCTGCTCACCGCCGCGCACCTGCTCTTTGTGGCGCCGCACAACGACTCGGCCACCGGGGAGCCCGAGGCGTCCTCGGGCTCGGGGCCGCCTTCCGGGGGCGCACTGGGGCCTCGGGCGCTGTTCGCCAGCCGCGTGCGCCCGGGCCAGCGCGTGTACGTGGTGGCCGAGCGTGACGGGGACCGCCGGCTCCTGCCCGCCGCTGTGCACAGCGTGACCCTAAGCGAGGAGGCCGCGGGCGCCTACGCGCCGCTCACGGCCCAGGGCACCATTCTCATCAACCGGGTGCTGGCCTCGTGCTACGCGGTCATCGAGGAGCACAGCTGGGCGCACCGGGCCTTCGCGCCCTTCCGCCTGGCGCACGCGCTCCTGGCTGCACTGGCGCCCGCGCGCACGGACCGCGGCGGGGACAGCGGCGGCGGGGACCGCGGGGGCGGCGGCGGCAGAGTAGCCCTAACCGCTCCAGGTGCTGCCGACGCTCCGGGTGCGGGGGCCACCGCGGGCATCCACTGGTACTCGCAGCTGCTCTACCAAATAGGCACCTGGCTCCTGGACAGCGAGGCCCTGCACCCGCTGGGCATGGCGGTCAAGTCCAGCTGAAGCCGGGGGGCCGGGGGAGGGGCGCGGGAGGGGGCC

SEQ ID NO: 2 - protein, 515 aa, Homo sapiens
AACAAGTTGGTTGGATACTTGCTGGAAAAAAAAAAGCAGTTTTAATGGTATTCAAAATACCTTTTAAAAAGTATTCTAGCACAAGATTTTTCTGTAAACTAGATTATGTTGTAAACTTTTTTCTAAATCTTGTAGGAGTGTCGGTTGTTAAGAACTAGAGCTTATTCCTATTCCAAATCTATCTTGCGCTCCTGAAAAACTGCAGAAAGGCACTTGAAAGCTGTTTCTTTAAGATATGGATTTCTTTTTTATTCTTGCTGGNAATATATTGCTGCACTGAGTGTGTGCAATTTTTATTCAAGGTCATCGTGATGCTGAGGAAGTTTCGTTGATAACCTGTCCCATCTCTAGGTTTCAACCGTCTTAATCAGAAGTGTCCNTTTTGAGTGGGGTATCCAACCAGAGGGGAGTGAATCCNGATTAGGTTTCCCTCAGGGTCAGGGGGGGNAAAGTTTGGGAGAGGCNGAGAAATCCNGCTCTCCTCGCCTTCCCGCAGCTTCCCGTAGGGGGGCCAT

SEQ ID NO: 3 - protein, 315 aa, Homo sapiens
ATGTCTCCCGCCCGGCTCCGGCCCCGACTGCACTTCTGCCTGGTCCTGTTGCTGCTGCTGGTGGTGCCGGCGGCATGGGGCTGCGGGCCGGGTCGGGTGGTGGGCAGCCGCCGGCGACCGCCACGCAAACTCGTGCCGCTCGCCTACAAGCAGTTCAGCCCCAATGTGCCCGAGAAGACCCTGGGCGCCAGCGGACGCTATGAAGGCAAGATCGCTCGCAGCTCCGAGCGCTTCAAGGAGCTCACCCCCAATTACAATCCAGACATCATCTTCAAGGACGAGGAGAACACAGGCGCCGACCGCCTCATGACCCAG

SEQ ID NO: 4 - protein, 283 aa, Homo sapiens
CCTCTCCCAGCGCTGCAAGGACCGCCTGAACTCGCTGGCTATCTCGGTGATGAACCAGTGGCCCGGTGTGAAGCTGCGGGTGACCGAGGGCTGGGACGAGGACGGCCACCACTCAGAGGAGTCCCTGCATTATGAGGGCCGCGCGGTGGACATCACCACATCAGACCGCGACCGCAATAAGTATGGACTGCTGGCGCGCTTGGCAGTGGAGGCCGGCTTTGACTGGGTGTATTACGAGTCAAAGGCCCACGTGCATTGCTCCGTCAAGTCCGGTGAGCCGCCG

SEQ ID NO: 5 - protein, 1481 aa, Homo sapiens
  N (315)..(415): N=GAP
ATGTCTCCCGCCCGGCTCCGGCCCCGACTGCACTTCTGCCTGGTCCTGTTGCTGCTGCTGGTGGTGCCGGCGGCATGGGGCTGCGGGCCGGGTCGGGTGGTGGGCAGCCGCCGGCGACCGCCACGCAAACTCGTGCCGCTCGCCTACAAGCAGTTCAGCCCCAATGTGCCCGAGAAGACCCTGGGCGCCAGCGGACGCTATGAAGGCAAGATCGCTCGCAGCTCCGAGCGCTTCAAGGAGCTCACCCCCAATTACAATCCAGACATCATCTTCAAGGACGAGGAGAACACAGGCGCCGACCGCCTCATGACCCAGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCTCTCCCAGCGCTGCAAGGACCGCCTGAACTCGCTGGCTATCTCGGTGATGAACCAGTGGCCCGGTGTGAAGCTGCGGGTGACCGAGGGCTGGGACGAGGACGGCCACCACTCAGAGGAGTCCCTGCATTATGAGGGCCGCGCGGTGGACATCACCACATCAGACCGCGACCGCAATAAGTATGGACTGCTGGCGCGCTTGGCAGTGGAGGCCGGCTTTGACTGGGTGTATTACGAGTCAAAGGCCCACGTGCATTGCTCCGTCAAGTCCGGTGAGCCGCCGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTTTCTCCCACAGAGCACTCGGCCGCAGCCAAGACGGGCGGCTGCTTCCCTGCCGGAGCCCAGGTACGCCTGGAGAGTGGGGCGCGTGTGGCCTTGTCAGCCGTGAGGCCGGGAGACCGTGTGTTGGCCATGGGGGAGGATGGGAGCCCCACCTTCAGCGATGTGCTCATTTTACTGGACCGCGAGCCCCACAGGCTGAGAGCCTTCCAGGTCATCGAGACTCAGGACCCCCCACGCCGCCTGGCACTCACACCCGCTCACTTGCTCTTTACGGCTGACAATCACACGGAGCCGGCAGCCCGCTTCCGGGCCACATTTGCCAGCCACGTGCAGCCTGGCCAGTACGTGCTGGTGGCTGGGGCGCCAGGCCTGCAGCCTGCCCGCGTGGCAGCTGTCTCTACACACGTGGCCCTCGGGGCCTACGCCCCGCTCACAAAGCATGGGACACTGGTGGTGGAGGATGTGGTGGCATCCTGCTTCGCGGCCGTGGCTGACCACCACCTGGCTCAGTTGGCCTTCTGGCCCCTGAGACTCTTTCACAGCTTGGCATGGGGCAGCTGGACCCCGGGGGAGGGTGTGCATTGGTACCCCCAGCTGCTCTACCGCCTGGGGCGTCTCCTGCTAGAAGAGGGCAGCTTCCACCCACTGGGCATGTCCGGGGCAGGGAGCTGAAAGGACTCCACC

SEQ ID NO: 6 - protein, 1567 aa, Gallus gallus
GAATTCGCGGCCGCATCAGACCCTTTTTGTCTCTCGATTGCTGTCTCCCGACCAAACTCCGATGTGTTCCGTTATCAGCGACCTAAAGGCTGCCATTCCAGCACCTGTCTTGCTAGGGATCGGTGGATAGTATACGATCAGAAAGCAGACAAGGACATAGGAGGAGAGAGAGCTCTAGAGAGACAGCCAGGGATAGGCACAGAGAGCTGTGAAGTAATTGGATTCAATGGTCGAAATGCTGCTGTTGACAAGAATTCTCTTGGTGGGCTTCATCTGCGCTCTTTTAGTCTCCTCTGGGCTGACTTGTGGACCAGGCAGGGGCATTGGAAAAAGGAGGCACCCCAAAAAGCTGACCCCGTTAGCCTATAAGCAGTTTATTCCCAATGTGGCAGAGAAGACCCTAGGGGCCAGTGGAAGATATGAAGGGAAGATCACAAGAAACTCCGAGAGATTTAAAGAACTAACCCCAAATTACAACCCTGACATTATTTTTAAGGATGAAGAGAACACGGGAGCTGACAGACTGATGACTCAGCGCTGCAAGGACAAGCTGAATGCCCTGGCGATCTCGGTGATGAACCAGTGGCCCGGGGTGAAGCTGCGGGTGACCGAGGGCTGGGACGAGGATGGCCATCACTCCGAGGAATCGCTGCACTACGAGGGTCGCGCCGTGGACATCACCACGTCGGATCGGGACCGCAGCAAGTACGGAATGCTGGCCCGCCTCGCCGTCGAGGCCGGCTTCGACTGGGTCTACTACGAGTCCAAGGCGCACATCCACTGCTCCGTCAAAGCAGAAAACTCAGTGGCAGCGAAATCAGGAGGCTGCTTCCCTGGCTCAGCCACAGTGCACCTGGAGCATGGAGGCACCAAGCTGGTGAAGGACCTGAGCCCTGGGGACCGCGTGCTGGCTGCTGACGCGGACGGCCGGCTGCTCTACAGTGACTTCCTCACCTTCCTCGACCGGATGGACAGCTCCCGAAAGCTCTTCTACGTCATCGAGACGCGGCAGCCCCGGGCCCGGCTGCTACTGACGGCGGCCCACCTGCTCTTTGTGGCCCCCCAGCACAACCAGTCGGAGGCCACAGGGTCCACCAGTGGCCAGGCGCTCTTCGCCAGCAACGTGAAGCCTGGCCAACGTGTCTATGTGCTGGGCGAGGGCGGGCAGCAGCTGCTGCCGGCGTCTGTCCACAGCGTCTCATTGCGGGAGGAGGCGTCCGGAGCCTACGCCCCACTCACCGCCCAGGGCACCATCCTCATCAACCGGGTGTTGGCCTCCTGCTACGCCGTCATCGAGGAGCACAGTTGGGCCCATTGGGCCTTCGCACCATTCCGCTTGGCTCAGGGGCTGCTGGCCGCCCTCTGCCCAGATGGGGCCATCCCTACTGCCGCCACCACCACCACTGGCATCCATTGGTACTCACGGCTCCTCTACCGCATCGGCAGCTGGGTGCTGGATGGTGACGCGCTGCATCCGCTGGGCATGGTGGCACCGGCCAGCTGAGAGTGGCTGGAGAGGGACAGAGACAGAGTGTGGGAGAGAGAAAAAAAAAGCGGCCGCGAATTC

SEQ ID NO: 7 - DNA, 20 bp, ARTIFICIAL
  CUSTOM SEQUENCE-PCR PRIMER
cacagcagcaagcagggatc

SEQ ID NO: 8 - DNA, 20 bp, ARTIFICIAL
  CUSTOM SEQUENCE-ARTIFICIAL PRIMER
ccgcggaacctgagaacttg

SEQ ID NO: 9 - DNA, 19 bp, ARTIFICIAL
  CUSTOM SEQUENCE PCR PRIMER
cggaacgcaaatatcgcac

SEQ ID NO: 10 - DNA, 28 bp, ARTIFICIAL
  CUSTOM SEQUENCE PCR PRIMER
gtgatccctctactttttcttctgactt

SEQ ID NO: 11 - DNA, 26 bp, ARTIFICIAL
  CUSTOM SEQUENCE PCR PRIMER
gagactagtgagacgtgctacttcca

(19) United States
(12) Patent Application Publication
(10) Pub. No.: US 2009/0305987 A1
(43) Pub. Date: Dec. 10, 2009

(54) Method For Inducing Beta Cell Neogenesis From Epithelial Cells

(75) Inventors: Sarah P. Thayer, Boston, MA (US); Jennifer LaFemina Fowler, Jamaica Plain, MA (US); Andrew L. Warshaw, Boston, MA (US); Carlos Fernandez-del Castillo, Newton, MA (US)

Correspondence Address: QUARLES & BRADY LLP, 411 E. WISCONSIN AVENUE, SUITE 2040, MILWAUKEE, WI, 53202-4497, US

(73) Assignee: The General Hospital Corporation, Boston, MA (US)

(21) Appl. No.: 12/467,707

(22) Filed: May 18, 2009

Related U.S. Application Data
(60) Provisional application No. 61/128,078, filed on May 19, 2008.

(51) Int. Cl. A61K 38/16; C12N 5/06

(52) U.S. Cl. 514/12; 435/375

(57) ABSTRACT

The invention provides a method of inducing β-cell neogenesis from epithelial cells. The method includes the step of exposing epithelial cells that have a disrupted G1-S cell cycle transition to a hedgehog protein in an amount effective to stimulate β-cell neogenesis from the epithelial cells. Cells resulting from the method are insulin-positive and express pancreatic progenitor cell markers including Pdx-1 and ngn3. β-cell population expansion is observed when the method is carried out in either in vitro or in vivo settings.

### CROSS REFERENCE TO RELATED APPLICATIONS

The present application claims the benefit of U.S. Provisional Application No. 61/128,078, filed May 19, 2008, which is incorporated herein by reference in its entirety for all purposes.

### STATEMENT REGARDING FEDERALLY SPONSORED RESEARCH OR DEVELOPMENT

The present invention was made with government support from NIH/NIDDK, grant no. 5K08DK071329-04. The United States Government has certain rights in this invention.

### FIELD OF THE INVENTION

The invention relates to methods of preventing and managing diabetes. In particular, the invention provides methods of inducing β-cell neogenesis from epithelial cells.

### BACKGROUND OF THE INVENTION

Diabetes mellitus is a severely debilitating disease, and β cell neogenesis has long been thought of as a potential means by which therapies and ultimately, cures, might be designed. Under normal conditions, β cell homeostasis remains in a fine balance, in which the number of p cells is determined by the rates of cellular renewal and destruction.

Cell renewal potentially arises from a number of mechanisms: β cell self-duplication and differentiation from a multipotential resident progenitor cell population in the pancreas. The embryological development of the pancreas occurs in a predictable, step-wise progression. Throughout the differentiation process, cells express specific markers, destining them to an endocrine or exocrine phenotype. Pdx-1 (also known as IDX-1, IPF-1, and STF-1) is transiently expressed as early as E8.5 and is necessary for the differentiation of pancreatic progenitor cells into the ductal, acinar, and islet cell lineages. Later, persistent Pdx-1 expression in the adult pancreas is limited to the islet and is necessary for both β cell differentiation and function. Ngn3 expression initiates at E9.0-9.5 and allows for the differentiation of endocrine lineage.

Genetic lineage models have demonstrated that under normal conditions, postnatal replenishment of the β cell population originates largely from pre-existing β cells rather than from a resident pluripotential cell population. However, in injury models, numerous studies have identified islet cells either in or in close proximity to the ductal epithelium, suggesting that facultative stem cells might reside in the ductal epithelium. Additionally, animals subject to partial pancreatectomy demonstrate increased ductal expression of Pdx-1 preceding regeneration of the endocrine compartment. It is possible that under abnormal conditions and with the proper external stimulus, adult ductal cells might in fact lose their terminally differentiated ductal phenotype and convert to a ductally-located, multipotential cell capable of β cell neogenesis via mechanisms reminiscent of the normal embryological development of β cells.

One such potential group of stimuli are the members of the hedgehog (Hh) signalling pathway. β cells are known targets of active Hh signaling. Indian hedgehog (Ihh), as well as downstream mediators of the Hh signaling pathway, are expressed within β cells. Though the role of Ihh within the islet and in β cell renewal is not fully elucidated, it is possible that hedgehog ligands, including Ihh, are involved in the proper functioning of β cells. Sonic hedgehog (Shh) specifically has been shown to play a role in the stimulation and maintenance of various stem cell niches, including neural and epidermal stem cells. Additionally, with the increasing support for stem cell contribution to tumorigenesis, Shh has been implicated in connection with the so-called “cancer stem cells” associated with multiple myeloma as well as breast and pancreatic cancers. While there is some evidence that the lack of Shh expression in the embryonic pancreas might result in increased islet density and endocrine cell type, it would be beneficial to identify if, in the adult pancreas, Shh signaling is involved in β cell function and specifically, the positive regulation of insulin production, in a manner redundant to that of Ihh regulation.

While β cell renewal plays a critical role in populating the β cell population, slowing the rate of β cell destruction to create an overall positive balance of islet cells is equally as important. P16 levels increase with age, and p16 has been implicated in driving cells into senescence. Increased expression, similar to ageing, results in diminished islet proliferation. However, the lack of p16 allows for bypass of senescence, maintenance and proliferation of progenitor cell niches, and ultimately, enhanced β cell proliferation.

Accordingly, a need exists for a method of identifying whether a system that drives a pluripotential cell through the cell cycle, in combination with the external stimulus of hedgehog protein, would allow for the correct set of stimuli to recapitulate the normal embryological development of the islet and thereby, adult neogenesis of β cells to prevent and/or manage diabetes mellitus.

### SUMMARY OF THE INVENTION

As can be appreciated, the ability to regenerate or grow islets, and more specifically, β cells, de novo has long posed a technical challenge. In spite of abundant past work, a possible mechanism of β cell neogenesis remains elusive. Here, the present inventors demonstrate an exemplary model in which the combination of Shh misexpression with p16^{-/-} results in β cell neogenesis.

In vivo, a transgenic mouse model that overexpresses Shh in the setting of the p16^{-/-} confirms that the combination of Shh misexpression with p16^{-/-} results in increased synaptophysin and insulin areas in murine pancreata and expands these cell populations in the ductal epithelium. These findings are associated with increased epithelial expression of pancreatic progenitor cells markers including Pdx-1 and ngn3.

In vitro data of p16^{-/-} ductal cells exposed to recombinant Shh demonstrates the growth of insulin-positive cells, particularly cells that are positive for both wide-spectrum cytokeratin and insulin. Real time PCR for Ins1 and Ins2 confirms that the combination of Shh and p16−/− does facilitate for increased expression of insulin in adult ductal cells. Expansion of the β cell population in these models suggests that, in general, the combination of hedgehog misexpression with a disrupted G1-S cell cycle transition induces an epithelial to endocrine transition. These findings suggest a mechanism by which β cell neogenesis may be stimulated de novo in adult pancreata as a means of addressing diseases related to endocrine insufficiency.

Accordingly, the invention provides a method of inducing β-cell neogenesis from epithelial cells. Such a method includes steps of exposing epithelial cells that have a disrupted G1-S cell cycle transition to a hedgehog protein in an amount effective to stimulate β-cell neogenesis from the epithelial cells.

In another aspect, the invention provides a method of inducing pancreatic epithelium to form a primordial epithelium capable of regenerating pancreatic components, including islet, duct and acinar cells. Such a method includes steps of disrupting the G1-S cell cycle transition in the pancreatic epithelium and exposing the resultant epithelium to a hedgehog protein in an amount effective to induce the pancreatic epithelium to form a primordial epithelium capable of regenerating pancreatic components from the epithelial cells.

Various hedgehog proteins are useful in the invention including desert hedgehog, indian hedgehog, or sonic hedgehog. Methods according to the invention are applicable in either in vitro or in vivo settings.

In an alternate embodiment, epithelial cells that have a disrupted G1-S cell cycle transition contain or are engineered to contain a dysfunctional INK4a/ARF locus which results in the appropriate cell cycle disruption. However, epithelial cells useful in the invention may have permanent or transient disruptions in a variety of genes and gene products associated with G1-S cell cycle transition including, but not limited to, a dysfunctional p53, Rb, cyclin, cyclin-dependent kinase, cyclin-dependent kinase inhibitor, or combination thereof.

In another aspect, the invention provides a method of treating a deficiency of insulin in a patient. Such a method includes inducing β-cell neogenesis in pancreatic epithelial cells of the patient by: (a) disrupting G1-S cell cycle transition in the pancreatic epithelial cells of the patient; and (b) exposing the pancreatic epithelial cells to a hedgehog protein in an amount effective to stimulate β-cell neogenesis from the pancreatic epithelial cells wherein the level of insulin is raised in the patient containing the pancreatic epithelial cells having undergone β-cell neogenesis. The method is particularly applicable where the patient is afflicted with diabetes.

These and other features, objects and advantages of the present invention will become better understood from the description that follows. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

FIG. 1. Qualitative assessment of islet architecture. Islets of PdxShh animals are poorly formed 1-2 cells units, demonstrated with stains for insulin (B, F) at 100× magnification representative 1 and 3 months animals. However, compared to wildtype (A, E) and p16−/− (C, G) controls, islet architecture in the PdxShh;p16−/− animals (D, H) is maintained. In addition, cells positive for insulin are demonstrated in the ductal epithelium of PdxShh;p16−/− animals (D, arrowhead).

FIG. 2. Expansion of pluripotential cell population in the PdxShh;p16−/− model. Immunohistochemical stains for Pdx-1 of representative 3 month animals, demonstrated at 200× magnification, demonstrate a significantly increased expression of Pdx-1 in the ductal epithelium in PdxShh;p16−/− animals (D, arrowhead), relative to wildtype (A), PdxShh (B), and p16−/− (C) animals. Additionally, Pdx-1-positive islet cells are located in close proximity to the Pdx-positive ductal epithelium, as demonstrated in the PdxShh;p16−/− animals (D, short arrow). Immunohistochemical stains for ngn3 of representative 8 month animals, demonstrated at 200× magnification, demonstrate ngn3 expression in the ductal epithelium of the PdxShh;p16−/− animals (H), compared to a lack of expression in wildtype (E), PdxShh (F), and p16−/− (G) animals.

FIG. 3. Expansion of insulin-positive cells in the p16−/− ductal epithelium after Shh exposure. Cell counts demonstrate that, with recombinant Shh treatment, there is a 2-fold increase in WSCK-positive p16−/− ductal cells. However, there is significant, 6-fold increase in cells that are positive for insulin, with cells that are positive for both WSCK and insulin making up a significant proportion of these cells (A). Seen at 200× magnification, immunofluorescent stains for WSCK (red) and insulin (green) demonstrate a significantly increased number of double positive cells in p16−/− ductal cells treated with Shh (C), compared to untreated controls (B).

FIG. 4. Real time PCR for Ins1 and Ins2. Averaged quantitative real time PCR data from four independent trials reveals a 2.5 fold (250%) increased expression of the murine Ins1 gene in p16−/− ductal cells treated with Shh×192 hours, relative to untreated to controls. Similarly there is a 1.15-fold (15%) increase expression of Ins2 in the same cells.

FIG. 5. Increase in secreted insulin as compared to total protein, relative to basal (unexposed p16−/− cells). P16−/− adult ductal cells were exposed to recombinant Shh. The quantity of secreted insulin and total cellular protein, relative to p16−/− cells exposed to 0 nM Shh and normalized to their respective total cellular protein, were assessed using EIA and protein assays. Cells exposed to 30 nM of recombinant Shh demonstrated a significant 2.4-fold increase in secreted insulin (P=0.039)

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” are intended to be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. Definitions

In describing the present invention, the following terms will be employed and are intended to be defined as indicated below.

As used herein, the term “hedgehog gene” refers to known members of the hedgehog gene family include sonic, indian, and desert hedgehog. A hedgehog gene or protein sequence useful in the invention may vary from one of the known sequences as long as it is at least 90% homologous at the nucleotide level over the complete gene sequence or has at least 80% sequence identity at the amino acid level over the complete amino acid sequence, and it retains the ability to stimulate the production of β cells or cells that produce insulin. A hedgehog variant may include deletion, insertion, or point mutants of hedgehog. Preferably a hedgehog variant will be able to hybridize to the known polynucleotide sequences provided herein under stringent conditions.

As herein used, the terms “stringent conditions” and “stringent hybridization conditions” mean hybridization occurring only if there is at least 95% and preferably at least 97% identity between the sequences, and is established via overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 micrograms/ml of denatured, sheared salmon sperm DNA, followed by washing the hybridization support in 0.1×SSC at about 65° C., as described in Sambrook, et al., Molecular Cloning: A Laboratory Manual, Second Edition, Cold Spring Harbor, N.Y., (1989), particularly Chapter 11 therein.

As used herein, a “therapeutically effective amount” with respect to hedgehog gene or protein refers to the amount of hedgehog gene or protein that is necessary to restore the level of insulin in the body to a normal level.

As used herein, a “normal” or “effective” level of endogenous insulin in a patient refers to the level of insulin produced endogenously in a healthy patient, i.e., a patient who is not afflicted with diabetes mellitus; i.e., in the range of 2-20 microunits/ml during fasting and 50-100 microunits/ml during non-fasting. This amount may vary in each healthy individual but it will be an amount sufficient to avoid the symptoms of diabetes mellitus. The normal levels of insulin often are indicated by a normal level of glucose in the blood under fasting conditions, i.e., a plasma glucose value of 70-110 mg/dL, and thus a variance from the normal amount of blood sugar also may be used to indicate whether a normal amount of insulin is produced.

As used herein, “insulin production” refers to expression of the insulin gene, insulin synthesis and/or insulin secretion by pancreatic β-cells.

As used herein, “deficiency of insulin” refers to the reduced levels of insulin as compared to the levels of insulin produced endogenously in a healthy patient, i.e., a patient who is not afflicted with diabetes. As used herein, “reduced” refers to up to 90%, 80%, 70%, 60%, 50%, 40%, 30%, 20% or 10% of average insulin levels associated with patients that have diabetes mellitus (which is also characterized by symptoms including hyperglycemia, glycosuria, polyuria, polydypsia, ketonuria, insulin resistance, and/or rapid weight loss) as compared to the levels of insulin produced endogenously in a healthy patient, i.e., a patient who is not afflicted with diabetes. A deficiency of insulin may be indicated by the range of 2-20 microunits/ml insulin for a patient under non-fasting conditions, or by a fasting plasma glucose value of ≧140 mg/dL, or an oral glucose tolerance test plasma glucose value of ≧200 mg/dL, or an elevated blood hemoglobin A₁ C(HGA₁ C) of ≧6.4%.

As used herein, “β cells” or “beta cells” refer to differentiated insulin-producing β cells.

As used herein, “PDX-1” refers to the gene which also has the synonyms/acronyms IPF-1, IDX-1, IUF-1, GSF-1, and STF-1.

As used herein, “β cell neogenesis” refers to the process of the differentiation of new cells from precursor cells, including pancreatic ductal precursors.

As used herein, “patient” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “patient” does not denote a particular age or sex. As used herein, “administering” or “administration” includes any means for introducing a compound into the body, preferably into the systemic circulation. Examples include but are not limited to oral, buccal, sublingual, pulmonary, transdermal, transmucosal, as well as subcutaneous, intraperitoneal, intravenous, and intramuscular injection.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. The terms embrace both preventative, i.e., prophylactic, and palliative treatment. Treating includes the administration of a compound of the present invention to prevent the onset of the symptoms or complications, to alleviate the symptoms or complications, or to eliminate the disease, condition or disorder.

A therapeutically effective amount of a compound may be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. It is also noted that the dose of the compound can be varied over time. A compound can be administered using an immediate release formulation, a controlled release formulation, or combinations thereof. The term “controlled release” includes sustained release, delayed release and combinations thereof.

A pharmaceutical composition of the invention can be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

### III. The Invention

The present invention utilizes epithelial cells in which the G1-S cell cycle transition is disrupted to recapitulate the normal embryological development of the islet cells and thereby, adult neogenesis of β cells to prevent and/or manage diabetes mellitus.

A “disrupted G1-S cell cycle transition” refers to a G1-S cell cycle transition that has been artificially interrupted from its expected natural progression as compared to the G1-S cell cycle transition observed for normal epithelial cells of like kind under similar growth conditions absent the artificial interruption. It is understood that the mammalian cell cycle control system is regulated by a group of cell cycle regulators, which include “cell cycle proteins” such as, e.g., protein kinases called “cyclin-dependent kinases” (CDKs). CDKs catalyze the attachment of phosphate groups to specific serine or threonine amino acids in a target protein. The phosphate groups alter the target protein's properties, such as its interaction with other proteins. (The alteration of protein activity by the attachment of phosphate groups occurs frequently in cells.)

CDKs are termed “cyclin-dependent” because their activity requires their association with activating subunits referred to as “cyclins”. While the number of CDKs in a cell remains constant during the cell cycle, the levels of cyclins oscillate. There are G1 cyclins, S-phase cyclins, and G2/M cyclins, each of which interact differently with CDK subunits to regulate the various phases of the cell cycle. CDKs can also associate with inhibitory subunits called CDK inhibitors (CKIs). In response to signals that work against proliferation, such as growth factor deprivation, DNA damage, cell-cell contact inhibition and lack of cell adhesion, CKIs cause the cell cycle to halt.

Many structurally related cyclins (A1, A2, B1, B2, B3, B4, B5, C, D1, D2, D3, E1, E2, F, G1, G2, H, I, L, and T) and nine CDKs (CDK1 to CDK9) have been identified in mammalian cells. Complexes of cyclin D and CDK4, as well as complexes of cyclin D and CDK6, operate during the G1 phase. Complexes of cyclin A and CDK2, as well as complexes of cyclin E and CDK2, act during the transition from the G1 to the S phase. Complexes of cyclin A and CDK1, as well as cyclin B and CDK1, function during the transition from the G2 to the M phase.

Active complexes of cyclins and CDKs exert their biological effects by phosphorylating proteins. During the G1 phase, a major target of cyclin/CDK complexes is the retinoblastoma protein (“Rb”). Rb is a growth-suppressing protein whose activity is controlled by whether or not it is phosphorylated (termed “pRb”). Rb is in the dephosphorylated form, during the G0 phase and early in the G1 phase, it is active. pRb exerts its growth-suppressing effects by binding to many cellular proteins, including the transcription factors of the E2F family. E2F transcription factors regulate the expression of numerous genes that are expressed during G1, or at the transition from the G1 to the S phase, to initiate DNA replication. pRb that is bound to an E2F transcription factor inhibits the transcription factor's activity. Following phosphorylation by cyclin/CDK complexes, pRb dissociates from E2F, allowing the transcription factor to bind DNA sequences and activate the expression of genes necessary for the cell to enter the S phase. Cyclin D1/CDK4 complexes phosphorylation of pRb during the middle of the G1 phase. They allow for subsequent phosphorylation of pRb by additional cyclin/CDK complexes that act later in the cell cycle.

At least two families of CKIs have been identified, based on their amino acid sequence similarity and the specificity of their interactions with CDKs. One of the families of CKIs, the INK family, includes four proteins (p15, p16, p18 and p20). These CKIs exclusively bind complexes of cyclin D and CDK4, as well as complexes of cyclin D and CDK6, to block cells that are in the G1 phase of the cell cycle. The other family of CKIs, the Cip/Kip family, consists of three proteins (p21, p27, and p57). These inhibitors bind to all complexes of cyclins and CDKs that function during the G1 phase and during the transition from the G1 to the S phase. They act preferentially, however, to block the activity of complexes containing CDK2.

Accordingly, the present invention utilizes epithelial cells in which one or more cell cycle regulator(s) is either permanently or transiently rendered dysfunctional to provide epithelial cells that have a disrupted G1-S cell cycle transition. As used herein, the term “dysfunctional” shall encompass a deficiency in a respective gene's expression level or a deficiency in an expressed product's biological activity, whether it be partial or complete deficiency, as it relates to G1-S cell cycle transition. Such epithelial cells provide the appropriate setting in which mis-expression of hedgehog protein then induces the epithelial cells to form primordial epithelium capable of regenerating various components of the pancreas, including acinar and islet cells (i.e., β cell neogenesis).

“Primordial epithelial cells” are defined herein as those cells capable of regenerating at least acinar and β cell components of the pancreas. A particularly preferred embodiment of the invention utilizes epithelial cells in which the INK4a/ARF locus (p16 and p19 (in mouse) or p16 and p14 (in human), is rendered dysfunctional thereby providing epithelial cells having the desired disruption in G1-S cell cycle transition.

DNA encoding hedgehog protein, tumor suppressor and cell cycle regulators described herein may be cDNA or genomic DNA. As known in the art, cDNA sequences have the arrangement of exons found in processed mRNA, forming a continuous open reading frame, while genomic sequences may have introns interrupting the open reading frame. Nucleic acids useful in the invention shall be intended to mean the open reading frame encoding a specific polypeptide but not adjacent 5′ and 3′ non-coding nucleotide sequences involved in the regulation of expression; the latter sequences are called regulating sequences that are operatively associated with the gene in its native state or are operative in a recombinant state useful in the invention.

The mammalian hedgehog family consists of sonic (Shh), indian (Ihh), and desert (Dhh) hedgehog proteins or other members of the hedgehog family as yet not identified or discovered. The known genes and gene products (also referred to as coding sequence) are provided below for the human hedgehogs. The gene sequence for the human Shh, is available at GenBank Accession No. L38518 (SEQ ID NO: 1) and its gene product, corresponding to nucleotides 152-1540. The available sequences of mammalian Dhh proteins include partial coding sequences for rat and human Dhh, and a complete sequence for mouse Dhh. The partial human Dhh gene and coding sequences are available at GenBank Accession No. U59748 (translated from nucleotides 1-285) (SEQ ID NO: 2). The sequences available for the Ihh proteins include a complete coding sequence for human Ihh derived from three exons, a complete sequence for mouse Ihh, and a partial coding sequence for rat Ihh. The sequences for the human Ihh exon 1 (SEQ ID NO: 3-GenBank Accession No. AB010092), exon 2 (SEQ ID NO: 4-GenBank Accession No. AB018075), and exon 3 (SEQ ID NO: 5-GenBank Accession No. AB018076) are respectively available in GenBank.

The hedgehog sequence information provided above can be used to obtain nucleic acid encoding a hedgehog gene through a number of methods familiar to those of skill in the art. For instance, nucleic acid encoding a hedgehog gene can be amplified from a complementary DNA (cDNA) library with the polymerase chain reaction (PCR). In this case, synthetic oligonucleotide primers directed to the 5′ and 3′ ends of a given hedgehog sequence can be generated based on the sequence data provided. The primers can then be used in conjunction with a cDNA library, which can be purchased from commercial suppliers such as Stratagene (La Jolla, Calif.), to amplify a cDNA sequence between and including the primer sequences, thereby providing the nucleic acid encoding a hedgehog gene from a cDNA library. The amplified cDNA can then be cloned into any of a number of plasmid vectors, such as those that enable protein expression or the generation of nucleic acids that can be used as probes for nucleic acids that encode hedgehog. Once cloned into a plasmid vector, a substantial quantity of the nucleic acid encoding a hedgehog gene can be obtained by propagation of the vector according to conventional techniques.

As briefly described, cDNA corresponding to a hedgehog gene can be isolated for human hedgehog as described by Marigo et al., 1995, Genomics 28:441 (herein incorporated by reference for all purposes), in which two human hedgehog homologs, Shh and Ihh, were cloned. Sequence comparison of several hedgehog genes, including mouse Shh, Ihh, and Dhh, and chick Shh (SEQ ID NO: 6-GenBank Accession No. L28099) showed that several regions within the second exon are apparently invariant among genes of this family. Degenerate oligonucleotides directed to these regions are used to amplify human genomic DNA by nested PCR.

The expected 220-bp PCR product is subcloned into pGEM7zf (Promega, Madison, Wis.) and sequenced using Sequenase v2.0 (U.S. Biochemicals, Cleveland, Ohio). A clone displaying high nucleotide similarity to mouse Ihh and mouse Shh sequences (Echelard, et al., 1993, Cell 75: 1417) is used for screening a human fetal lung 5′-stretch plus cDNA library (Clontech, Palo Alto, Calif.) in λgt10 phage. The library is screened following the protocol suggested by the company, and positive plaques are identified, purified, subcloned into pBluescript SK(+) (Stratagene, La Jolla, Calif.), and sequenced, identifying them as the human homologues of Shh and Ihh.

The hedgehog gene sequences provided above may be employed to produce the hedgehog proteins useful in the invention. These proteins include intact hedgehog, or an active fragment thereof, since the bioactive signaling form of hedgehog may be derived by proteolysis of a protein precursor, as is the case for Shh. According to methods familiar to those of skill in the art, nucleic acid encoding the gene is cloned into an appropriate vector (such as described above and known to one of skill in the art) so that it may be expressed, and the protein is then produced by expression of the gene and subsequent purification of the expressed protein. For expression, an expression cassette may be employed, providing for a transcriptional and translational initiation region, which may be inducible or constitutive, the coding region under the transcriptional control of the transcriptional initiation region, and a transcriptional and translational termination region. Various transcriptional initiation regions may be employed which are functional in the expression host.

The peptide may be expressed in prokaryotes or eukaryotes by conventional techniques, depending upon the purpose for expression. For large scale production of the protein, a unicellular organism may be used as the expression host, such as E. coli, B. subtilis, and S. cerevisiae. Alternatively, cells of a multicellular organism, e.g. eukaryotes such as vertebrates, particularly mammals, may be used as the expression host. In many situations, it may be desirable to express the subject hedgehog gene in a mammalian host cell, whereby the hedgehog gene product is cholesterolated, and secreted. In fact, hedgehog proteins useful in the present invention may be augmented by a wide variety of enzymatic and chemical modifications known in the art, including but not limited to, the addition of fatty acids or a cholesterol moiety, and glycosylation.

With the availability of the protein in large amounts by employing an expression host, the protein may be isolated and purified by conventional techniques. A lysate may be prepared of the expression host and the lysate purified using HPLC, exclusion chromatography, gel electrophoresis, affinity chromatography, or other purification techniques. The purified protein will generally be at least about 80% pure, preferably at least about 90% pure, and may be up to and including 100% pure. As used herein, “pure” is intended to mean free of other proteins, as well as of cellular debris.

Hedgehog is delivered to the patient in an amount sufficient to provide an effective level of endogenous insulin in the patient. An “effective” or “normal” level of endogenous insulin in a patient refers generally to that level of insulin that is produced endogenously in a healthy patient, i.e., a patient who is not afflicted with diabetes. Alternatively, an “effective” level may also refer to the level of insulin that is determined by the practitioner to be medically effective to alleviate the symptoms of diabetes.

Methods of the invention include hedgehog administration in the form of hedgehog protein, nucleic acids encoding, a vector containing a hedgehog gene (gene therapy), epithelial cells expressing hedgehog protein, and tissue which contains epithelial cells expressing hedgehog protein thereby providing a primordial epithelium which is capable of regenerating to yield various pancreatic cells including, e.g., islet, duct and acinar cells.

Methods of the invention include administering hedgehog protein or a variant thereof so that epithelial cells having a disrupted G1-S cell cycle transition within the patient or alternatively, in a tissue sample for transplantation purposes, are induced to undergo β cell neogenesis. Thus, according to the invention, a diabetic patient may be treated by disrupting the G1-S cell cycle transition in pancreatic epithelium followed by administering a hedgehog protein, or a variant thereof to cause the affected epithelial cells to regenerate components of the adult pancreas, including insulin positive cells.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

Target cells useful according to the invention will include epithelial cells, including pancreatic epithelial cells (e.g., non-islet pancreatic cells, non-β-cell islet cells, and pancreatic duct cells) and epithelial cells of extra-pancreatic origin, all of said cells having a disruption of the G1-S cell cycle transition, which can be loss (complete or partial) or knockdown (complete or partial) of a tumor suppressor (e.g., p53 and Rb), cyclin, cyclin-dependent kinase, cyclin-dependent kinase inhibitor, or combination thereof. Particularly preferred genes for disruption are p16, p19, p21, p27, p53, and Rb with a particularly preferred combination of genes designated the INK4a/ARF locus (i.e., p16 and p19 in mouse/p16 and p14 in human).

Various methodologies may be utilized for disrupting, disabling, or silencing genes involved in G1-S cell cycle transition and include, but are not limited to, knockout or knockdown technologies (which can include, but are not limited to, siRNA, shRNA, RNAi) as well as pharmacologic manipulations (e.g., modulating the p16 cell cycle checkpoint). Additional options include permanent knockouts, which can include recombinant engineering, immunological therapies (e.g., antibodies), and modulating proteins that are known to bind to and interfere with the function of a specific target protein. Such methods can be applied, for example, by disrupting G1-S transition in epithelial cells in vitro and then transplanting the resulting cells into a human, with hedgehog treatment occurring before or after transplantation. Alternatively, directed gene therapy, in vivo, may achieve the knockdown of cell cycle regulators, e.g., INK4a/ARF products, with the subsequent expression of hedgehog protein to induce formation of primordial epithelium.

The patient may be treated with intact hedgehog protein, or an active fragment thereof, particularly a cleaved fragment as generated by normal processing. Desirably, the hedgehog peptides will not induce an immune response, particularly an antibody response. Xenogeneic analogs may be screened for their ability provide a therapeutic effect, most advantageously, without raising an immune response.

Various methods for administration may be employed. The polypeptide formulation may be given orally, or may be injected intravascularly, subcutaneously, intrapancreatically, peritoneally, and so forth. The dosage of the therapeutic formulation will vary widely, depending upon the frequency of administration, the manner of administration, and the clearance of the agent from the patient. For example, the dose may range from 1 to 10 mg hedgehog protein/kg body weight. The initial dose may be larger, followed by smaller maintenance doses. The dose may be administered as infrequently as weekly or biweekly, or fractionated into smaller doses and administered daily, semi-weekly, etc. to maintain an effective dosage level. In many cases, oral administration will require a higher dose than if administered intravenously.

The hedgehog peptides may be prepared as formulations at a pharmacologically effective dose in pharmaceutically acceptable media, for example normal saline, PBS, etc. The additives may include bacteriocidal agents, stabilizers, buffers, or the like. To enhance the half-life of the subject peptide or subject peptide conjugates, the peptides may be encapsulated, introduced into the lumen of liposomes, prepared as a colloid, or another conventional technique may be employed that provides for an extended lifetime of the peptides.

In addition to administering hedgehog protein, another means of administration according to the invention is administration of DNA encoding hedgehog or its variant. Thus, according to the invention, insulin deficiency may be treated by administering to a patient a nucleic acid encoding hedgehog or a variant thereof. The nucleic acid will optimally be carried by a vehicle, and is administered in numbers sufficient to provide an effective level of endogenous insulin in a patient.

The administration of nucleic acids encoding hedgehog to treat insulin deficiency is an application of gene therapy, which involves the direct manipulation and use of genes to treat disease. A hedgehog gene may be delivered to a target cell with relatively high specificity and efficiency according to methods known in the art. There are multiple ways to deliver and express genes as part of a gene therapy protocol. Typically, a nucleic acid of interest will be propagated and carried on an episomal vector.

The following gene therapy methods are representative of gene therapy methods useful for accomplishing gene therapy according to the invention, and are not limiting to the invention.

An episomal vector containing the therapeutic hedgehog gene or variants thereof (herein, an episomal vector containing the hedgehog gene will be referred to as the hedgehog vector) can be administered to patients harboring pancreatic epithelium that has been engineered to exhibit a disrupted G1-S cell cycle transition in order to treat a deficiency of insulin. The hedgehog gene may be delivered by, e.g., exogenous delivery of a naked hedgehog vector, a hedgehog vector associated with specific carriers, by means of an appropriate delivery vehicle, e.g., a liposome, by use of iontophoresis, electroporation and other pharmacologically approved methods of delivery. Routes of administration may include intramuscular, intravenous, aerosol, oral (tablet or pill form), topical, systemic, ocular, as a suppository, intrapancreatic, intraperitoneal and/or intrathecal.

In terms of disrupting cell cycle regulators (at the gene, mRNA, and/or protein level) in a patient and, as well, providing hedgehog to a patient, there are multiple ways to deliver nucleic acids as part of a gene therapy protocol. At least three types of delivery strategies are useful in the present invention, including: injection of naked vector, or injection of charge modified vector, or particle carrier drug delivery vehicles. Unmodified nucleic acid, like most small molecules, are taken up by cells, albeit slowly. To enhance cellular uptake, the vector may be modified in ways which reduce its charge but will maintain the expression of specific functional groups in the final translation product. This results in a molecule which is able to diffuse across the cell membrane, thus removing the permeability barrier.

Chemical modifications of the phosphate backbone may be used to reduce the negative charge allowing free diffusion across the membrane. In the body, maintenance of an external concentration of the vector relative to the pancreas will be necessary to drive the diffusion of the modified vector into the epithelial cells of the pancreas. Administration routes which allow the pancreas to be exposed to a transient high concentration of the nucleic acid which is slowly dissipated by systematic adsorption are preferred. Intravenous administration with a drug carrier designed to increase the circulation half-life of the vector can be used. The size and composition of the drug carrier restricts rapid clearance from the blood stream. The carrier, made to accumulate at the desired site of transfer, can protect the vector from degradative processes.

Drug delivery vehicles are effective for both systemic and topical administration. They can be designed to serve as a slow release reservoir, or to deliver their contents directly to the target cell. An advantage of using direct delivery drug vehicles is that multiple molecules are delivered per uptake. Such vehicles have been shown to increase the circulation half-life of drugs which would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The nucleic acid may also be systemically administered. Systemic absorption refers to the accumulation of drugs in the blood stream followed by distribution throughout the entire body. A gene gun may also be utilized to administer a particular vector. Administration of DNA-coated microprojectiles by a gene gun requires instrumentation but is as simple as direct injection of DNA. A construct bearing the gene of interest is precipitated onto the surface of microscopic metal beads. The microprojectiles are accelerated with a shock wave or expanding helium gas, and penetrate tissues to a depth of several cell layers. This approach permits the delivery of foreign genes to the skin of anesthetized animals. This method of administration achieves expression of transgenes at high levels for several days and at detectable levels for several weeks. Each of these administration routes exposes the vector to the targeted pancreas. Subcutaneous administration drains into a localized lymph node which proceeds through the lymphatic network into the circulation. The rate of entry into the circulation has been shown to be a function of molecular weight or size. The use of a liposome or other drug carrier localizes the vector at the lymph node. The vector can be modified to diffuse into the cell, or the liposome can directly participate in the delivery of either the unmodified or modified vector to the epithelial cell. Liposomes injected intravenously show accumulation in the liver, lung and spleen. The composition and size can be adjusted so that this accumulation represents 30% to 40% of the injected dose. The remaining dose circulates in the blood stream for up to 24 hours.

The dosage will depend upon the disease indication and the route of administration but should be between 1-1000 μg vector/kg of body weight/day. The duration of treatment will extend through the course of the disease symptoms, possibly continuously. The number of doses will depend upon disease delivery vehicle and efficacy data from clinical trials.

Another means of administration according to the invention entails administering cells that have a disrupted G1-S cell cycle transition and that express hedgehog protein. Thus, according to the invention, patients that have insulin deficiency may be treated by administering epithelial cells that have a disrupted G1-S cell cycle transition and that express hedgehog protein or a variant thereof. The cells are administered in numbers sufficient to provide an effective level of endogenous insulin in a patient.

In this approach, epithelial cells may be, for example, cultured in vitro and transfected with nucleic acids encoding the hedgehog gene and, in addition, an RNAi construct to partially or completely silence cell cycle regulatory proteins, such as, e.g., the products of the INK4a/ARF locus. The cell type used would be limited to those that would be compatible with systemic administration in patients, and thus presumably would be human epithelial cells, preferably those cultured from the patient to receive the administration. The epithelial cells would be transfected with the nucleic acids by means known in the art. The cells would then experience a disruption in G1-S cell cycle transition and also express the transfected hedgehog gene, producing hedgehog protein, thereby forming primordial pancreatic epithelium capable of regenerating adult pancreatic components, including insulin positive cells. Transfection of the cells can be monitored in several ways, including examining samples of the growth media with antibodies to hedgehog. After cells are successfully transfected and have been induced to yield primordial epithelium, they can be prepared for systemic administration to patients. To prepare cells for administration, they can be washed free of growth media, and placed in an appropriate pharmaceutically-acceptable media that is both compatible for the cells and the host patient.

To ensure expression of particular expression constructs in transfected cells, the expression of such constructs can be analyzed in vitro. For example, western blot analysis can be carried out to examine expression of Shh protein. For the Western blot analysis, whole-cell extracts are made from transduced and control β-cells and analyzed simultaneously with antibodies to human Shh as well as to B220 (CD45R) for an internal control. Approximately 20 μg of cell extract is analyzed by Western blotting. For examination of gene expression, mRNA is purified from whole-cell extracts and digested with deoxyribonuclease I (DNase I; Gibco BRL, Gaithersburg, Md.) to remove any contaminating genomic DNA. Reverse transcriptase polymerase chain reaction analysis is then performed with primers directed to Shh, and is internally controlled by examining expression of B220 by inclusion of B220 primers in all reactions.

The dosage of the therapeutic cells described herein will vary widely from about 1×10³ to 1×10⁷ cells per individual, depending upon the hedgehog expression levels of the cells, the degree of G1-S cell cycle disruption of the cells, the frequency of administration, the manner of administration, and the clearance of the agent from the patient. The initial dose may be larger, followed by smaller maintenance doses. The dose may be administered as infrequently as weekly or biweekly, or fractionated into smaller doses and administered daily, semi-weekly, etc. to maintain an effective dosage level that provides an effective level of endogenous insulin in the patient.

Another means of administration according to the invention is implantation of therapeutic cells as tissue. For example, patients that have diabetes mellitus type 1 are treated by administering epithelial tissue that has been induced to provide primordial pancreatic epithelium that will undergo β-cell neogenesis in the in vivo setting. Such tissue is administered in an amount sufficient to provide an effective level of endogenous insulin in the patient. For example, an ex vivo approach may be utilized, whereby pancreatic tissue is removed from a patient and administered vectors designed to silence the INK4a/ARF products in combination with the hedgehog gene so that the cells of the tissue are rendered capable of β-cell neogenesis. The tissue is then re-implanted into the patient (e.g., as described by Wilson, 1992, Hum. Gene Ther. 3:179) incorporated herein by reference for all purposes. The organized tissue is re-implanted into pancreas at the site from which it was removed or alternatively, adjacent to the pancreas. While the tissue can be temporarily implanted, it is preferred to permanently implant the organized tissue so as to establish an expanding population of cells capable of providing regeneration of pancreas components, including insulin positive β-cells and acinar cells.

In an alternate embodiment of the invention, a kit for inducing α-cell neogenesis from epithelial cells according to the present invention is provided. In one embodiment, the kit comprises epithelial cells that have a disrupted G1-S cell cycle transition, a hedgehog protein in an amount effective to stimulate β-cell neogenesis from said epithelial cells and instructions for use.

In an alternate embodiment of the invention, a kit for inducing pancreatic epithelium to form a primordial epithelium capable of regenerating pancreatic islet, duct and acinar cells according to the present invention is provided. The kit comprises epithelial cells having a disrupted G1-S cell cycle transition, a hedgehog protein in an amount effective to induce the pancreatic epithelium to form a primordial epithelium capable of regenerating pancreatic islet, duct and acinar cells from the pancreatic epithelium, and instructions for use.

In yet another embodiment of the invention, a kit for treating a deficiency of insulin in a patient is provided. The kit comprises epithelial cells having a disrupted G1-S cell cycle transition, a hedgehog protein in an amount effective to stimulate β-cell neogenesis from the pancreatic epithelial cells wherein, upon placement of the resulting β-cells in the patient, the level of insulin is raised in the patient containing the pancreatic epithelial cells having undergone β-cell neogenesis and instructions for use.

By “instructions for use” we mean a publication, a recording, a diagram, or any other medium of expression which is used to communicate the usefulness of the invention for one of the purposes set forth herein. The instructional material of the kit can, for example, be affixed to a container which contains the present invention or be shipped together with a container which contains the invention. Alternatively, the instructional material can be shipped separately from the container or provided on an electronically accessible form on a internet website with the intention that the instructional material and the kits contents be used cooperatively by the recipient.

The invention is illustrated by the following non-limiting examples wherein the following materials and methods are employed. The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1

### General Materials and Methods

### Generation and Maintenance of Transgenic and Bigenic Mice.

PdxShh mice were created as previously reported and backcrossed onto a C57BL/6J background using a C57BL/6J mouse (Jackson Laboratories; Bar Harbor, Me.). Bigenic mice were created by breeding PdxShh mice to a p16/p19^{-/-} FVB mouse (INK4a/ARF knockout mouse was a generous gift from R. DePinho) to create the PdxShh;p 16/p19^{-/-} bigenic mouse line. Wildtype and p16/p19^{-/-} mice served as controls for PdxShh and PdxShh;p16/p19^{-/-} mice, respectively. Timepoints were generated for all monthly timepoints from 1 to 12 months. Three-group analysis was conducted on the 1, 3, 5 and 8 month animals.

### Genotypic Confirmation of Transgenic and Bigenic Mice.

Genomic DNA was isolated from mouse tails of 6 week old pups. PCR was used to screen for transgenic and bigenic mice. Pdx-rat Shh PCR primers included: Pdx forward primer, 5′-CAC AGC AGC AAG CAG GGA TC-3′ (SEQ ID NO: 7); rat Shh reverse primer, 5′-CCG CGG AAC CTG AGA ACT TG-3′ (SEQ ID NO: 8). P16 PCR primers include p16 WT primer, 5′-CGG AAC GCA AAT ATC GCA C-3′ (SEQ ID NO: 9); p16 forward primer, 5′-GTG ATC CCT CTA CTT TTT CTT CTG ACT T-3′ (SEQ ID NO: 10); p16 reverse primer, 5′-GAG ACT AGT GAG ACG TGC TAC TTC CA-3′ (SEQ ID NO: 11).

PCR was carried out with a Robocycler Gradient 96 (Stratagene; La Jolla, Calif.). The PdxShh program was: 1 cycle of 5 min at 94° C., 35 cycles of 30 sec at 94° C. and 40 sec at 51° C. and 40 sec at 72° C., and 1 cycle of 5 min at 72° C. The p16 program was: 1 cycle of 5 min at 95° C., 35 cycles of 50 sec at 95° C. and 50 sec at 60° C. and 1 min at 72° C., and 1 cycle of 10 min at 72° C. PdxShh PCR products were run on a 1% agarose gel; p16 products were run on a 4% agarose gel.

### Phenotypic Analysis.

Animals were sacrificed at 1, 3, 5, and 8 months. Pancreata were harvested, washed in 1×PBS, and fixed overnight in 10% formalin. Immunohistochemistry for synaptophysin, insulin, and glucagon was performed on paraffin-embedded tissue. No antigen retrieval was used for insulin staining; antigen retrieval at pH 6 was used for synaptophysin and glucagon. Primary antibodies included synaptophysin (rabbit, 1:250; Abcam; Cambridge, Mass.), insulin (mouse IgG1, 1:100; Santa Cruz Biotechnology; Santa Cruz, Calif.), and glucagon (rabbit, 1:400; Abcam; Cambridge, Mass.). Secondary antibodies include biotinylated rabbit anti-mouse (1:1000 for insulin; Zymed; San Francisco, Calif.) and biotin-SP-conjugated AffiniPure F(ab′)₂ fragment goat anti-rabbit (1:500 for synaptophysin, 1:1000 for glucagon; Jackson ImmunoResearch Laboratories, West Grove, Pa.). Slides were developed with DAB and counterstained with hematoxylin. Slides were examined at 100× magnification with a Nikon Eclipse E600 microscope (Nikon). Photographs were taken were taken at 100× with a Nikon Photometrics CoolSnap Camera (Nikon).

Images were analyzed using MetaMorph (Molecular Devices; Downingtown, Pa.). For synaptophysin, insulin, and glucagon areas were calculated by randomly photographing at 100× the largest 6 islets or islet fields per pancreas. Three photographs were randomly selected and analyzed for synaptophysin, insulin, and glucagon area, as determine by a positive stain, using MetaMorph.

### Progenitor Cell Analysis.

Immunohistochemistry for Pdx-1 and ngn3 was performed on paraffin-embedded tissue. Antigen retrieval at pH 6 was used for Pdx-1; antigen retrieval citra (BioGenex; San Ramon, Calif.) was used for ngn3. Primary antibodies included Pdx-1 (guinea pig, 1:100; generous gift from C. Wright) and ngn3 (rabbit, 1:6000; generous gift from M. German). Secondary antibodies include biotinylated goat anti-guinea pig (1:500; Vector Laboratories; Burlingame, Calif.) and biotin-SP-conjugated AffiniPure F(ab′)₂ fragment goat anti-rabbit (1:500 for ngn3; Jackson ImmunoResearch Laboratories, West Grove, Pa.). Slides were developed with DAB and counterstained with hematoxylin. Slides were examined at 100× and 200× magnification with a Nikon Eclipse E600 microscope (Nikon; Melville, N.Y.). Photographs were taken were taken at 100× with a Nikon Photometrics CoolSnap Camera (Nikon).

Isolation and Creation of p16−/− Ductal Epithelial Cell Line.

P16−/− ductal epithelial cells were isolated from the pancreata of p16−/− transgenic mice with modifications from previous protocols. Cells were cultured in Dulbeccos' modified Eagle medium/F12 medium supplemented with D-glucose, insulin-transferrin-selenium, soybean trypsin, epidermal growth factor, bovine pituitary extract, 3,3′,5-triiodo-Lthyronine, cholera toxin, dexamethasone, nicotinamide, 5% Nu-serum IV culture supplement, amphotericin, penicillin, and streptomycin, per previous reports.

Shh Treatment of p16−/− Ductal Epithelial Cell Line.

Cells were plated onto a type I collagen-coated 60 mm Petri dishes or culture slides. After 12 hours, cells were exposed to 0 or 30 nM recombinant human Shh (R&D Systems, Inc.; Minneapolis, Minn.) in culture media. Media was changed every 48 hours. After 192 hours, cells were fixed for cell counts or RNA was isolated for real time PCR.

### In Vitro Cell Counts for Wide-Spectrum Cytokeratin and Insulin.

Cultured p16^{-/-} ductal epithelial cells exposed to 0 or 30 nM recombinant Shh×192 hours were paraformaldehyde-fixed for wide-spectrum cytokeratin (WSCK), insulin, and DAPI immunofluorescence. Primary antibodies included insulin (guinea pig; 1:100; Dako; Carpinteria, Calif.) and WSCK (rabbit; 1:100; Dako; Carpinteria, Calif.). Secondary antibodies included goat anti-rabbit IgG Alexa Fluor® 594 (1:500; Invitrogen Molecular Probes; Eugene, Oreg.) and goat anti-guinea pig IgG Alexa Fluor® 488 (1:500; Invitrogen Molecular Probes; Eugene, Oreg.). After incubation with secondary antibody, DAPI was applied. Slides were examined at 200× magnification with a Nikon Eclipse E600 microscope (Nikon). Photographs were taken were taken at 200× with a Nikon Photometrics CoolSnap Camera (Nikon). Five randomly selected merged images were counted for cells positive for DAPI, WSCK, insulin, and WSCK and insulin. Photographs were merged and analyzed using MetaMorph (Molecular Devices; Downingtown, Pa.). Experiments comparing 0 and 30 nM-treated cells were run in triplicate. Statistical analyses were performed using Student's paired t-tests with Microsoft Excel (Microsoft; Redmond, Wash.)

### Quantitative Real Time PCR for Insulin.

RNA was extracted (RNAqueous-4PCR Kit; Ambion; Austin, Tex.) from cultured p16−/− ductal epithelial cells exposed to 0 or 30 nM recombinant Shh×192 hours in four independent experimental runs. One-step multiplex TaqMan Real-time RT-PCR was performed using an ABI 7700 Sequence Detector system. Expression of murine Ins1 and Ins2 was evaluated; 18S RNA was used as the internal control. Probes and primers were designed within exon 2 of both genes (sequences are available on request). The thermal cycler conditions for these experiments were: reverse transcription for 30 min at 48° C., followed by 40 cycles of 10 min at 95° C. and 15 min at 95° C., and finally, 60 min at 60° C. Fluorescence data were collected during the annealing step and analyzed using SDS1.7 software (Applied Biosystems). Relative gene expression was determined based on corresponding Ct (threshold cycle) values.

### Example 2

### Shh Misexpression with p16−/− Stimulates in β Cell Neogenesis

Both Shh and p16^{-/-} have independently been shown to maintain of multipotential cell niches. The inventors in vivo data described in this example demonstrates that the combination of p16^{-/-} with increased Shh exposure might in fact augment the ability to generate such multipotential cell populations. In fact, it appears that the synergistic effect of these two factors drives homeostasis of β cell population towards a positive balance, favoring neogenesis and resulting in expansion of the β cell compartment in both the in vivo and in vitro settings. Prior studies have demonstrated that the absence of p16 modifies stem cell aging, and within the β cell compartment specifically, enhanced proliferation and regeneration. With the lack of p16 and the bypass of senescence, cells continue through the cell cycle. Then, with the addition of the proper set of stimuli, in this case, Shh overexpression, neogenesis of β cells—reminiscent of the normal embryological development of β l cells—may occur via pathways that are fundamental different from normal adult β cell renewal.

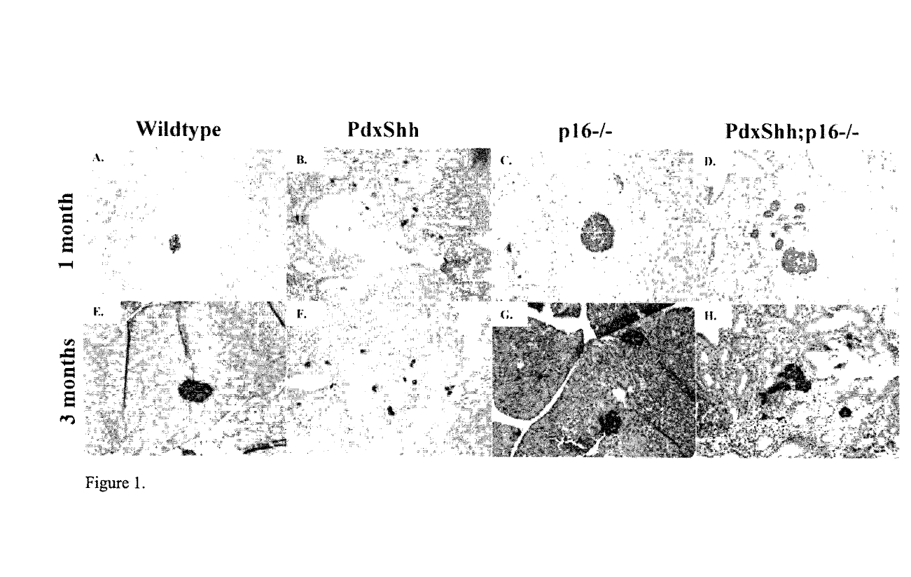

In comparing wildtype, PdxShh, p16^{-/-}, and PdxShh;p16^{-/-} mice at 1, 3, 5, and 8 months, PdxShh mice demonstrate highly atypical islets, whose architecture is defined by poorly formed, 1-2 cell units (FIGS. 1B, 1E). However, like wildtype (FIGS. 1A, 1E) and p16^{-/-} (FIGS. 1C, 1G) controls, islet architecture of PdxShh;p16−/− animals appears to be maintained (FIGS. 1D, H). When islets are quantitatively assessed via immunohistochemistry for synaptophysin, total synaptophysin area is significantly smaller in PdxShh animals compared to wildtype littermates at all timepoints (Table 1). Similarly, this area is also significantly smaller than age-matched PdxShh;p16^{-/-} animals at all timepoints. In contrast, total synaptophysin area of p16^{-/-} controls is equivalent to that of PdxShh;p16^{-/-} animals at all timepoints as well as to wildtype animals at 1, 3, and 5 months. When evaluating the relative contributions of endocrine cell types, the primary contributor of the increased islet area appears to be due to insulin-secreting cells, as the total insulin area in PdxShh;p16^{-/-} animals is significantly greater than age-matched PdxShh animals at 1, 3, and 5 months (Table 1). Similar to synaptophysin, insulin area in the p16^{-/-} animals is not only equivalent to that of the PdxShh;p16^{-/-} animals but also equivalent to wildtype. At all timepoints, the area of glucagon-secreting cells in PdxShh and PdxShh;p16^{-/-} animals is equivalent (Table 1).

TABLE 1
Phenotypic analysis of endocrine compartments.

| | Results of | | | | |
|---|---|---|---|---|---|
| | Analyzed | P value, | P value, | P value, | P value, |
| Hormone | Groups | 1 month | 3 month | 5 month | 8 month |
| Synaptophysin | WT > PdxShh | 0.0305 | 0.0011 | 0.0420 | 0.0003 |
| | WT > p16−/− | 0.3375 | 0.3457 | 0.4816 | 0.0014 |
| | PdxShh < | 0.0111 | 0.0215 | 0.0029 | 0.0318 |
| | PdxShh; p16−/− | | | | |
| | p16−/− > | 0.4272 | 0.4678 | 0.0548 | 0.2189 |
| | PdxShh; p16−/− | | | | |
| Insulin | WT > PdxShh | 0.0113 | 0.0373 | 0.0190 | 0.0022 |
| | WT > p16−/− | 0.5447 | 0.2451 | 0.1439 | 0.0089 |
| | PdxShh < | 0.0040 | 0.0511 | 0.0398 | 0.4371 |
| | PdxShh; p16−/− | | | | |
| | p16−/− > | 0.4228 | 0.7825 | 0.4386 | 0.0186 |
| | PdxShh; p16−/− | | | | |
| Glucagon | WT > PdxShh | 0.0026 | 0.0012 | 0.0175 | 0.0100 |
| | WT > p16−/− | 0.0111 | 0.0099 | 0.1425 | 0.5421 |
| | PdxShh < | 0.5089 | 0.0905 | 0.6083 | 0.7944 |
| | PdxShh; p16−/− | | | | |
| | p16−/− > | 0.0641 | 0.0383 | 0.1166 | 0.0685 |
| | PdxShh; p16−/− | | | | |

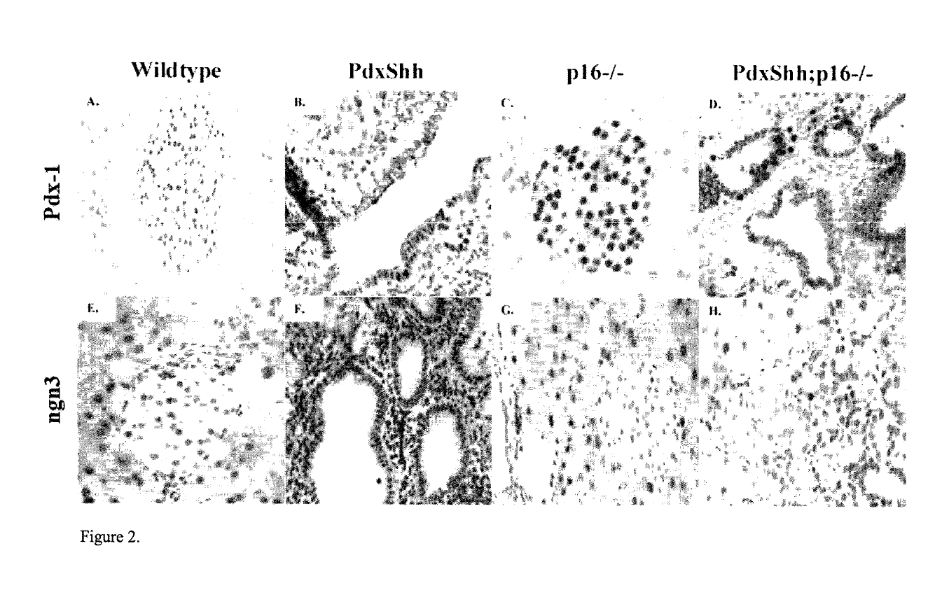

Pancreatic progenitor cells have the potential to give rise to multiple cell lineages and carry markers normally expressed during the embryologic development of the pancreas. Two such markers include Pdx-1 and ngn3, both of which have very limited expression in the wildtype, adult pancreatic ductal epithelium. Immunohistochemical stains for both Pdx-1 and ngn3 demonstrate an increased number of Pdx-1- and ngn3-positive cells in the ductal epithelium of PdxShh;p16^{-/-} pancreata, relative to both controls as well as PdxShh animals (FIG. 2). While it is not clear at this time if these cells are involved in the repopulation of β cells in the ductal epithelium, it does appear that the synergistic effect of Shh overexpression with p16^{-/-} allows for the stimulation of a pluripotential pancreatic progenitor population, reminiscent of embryologic development.

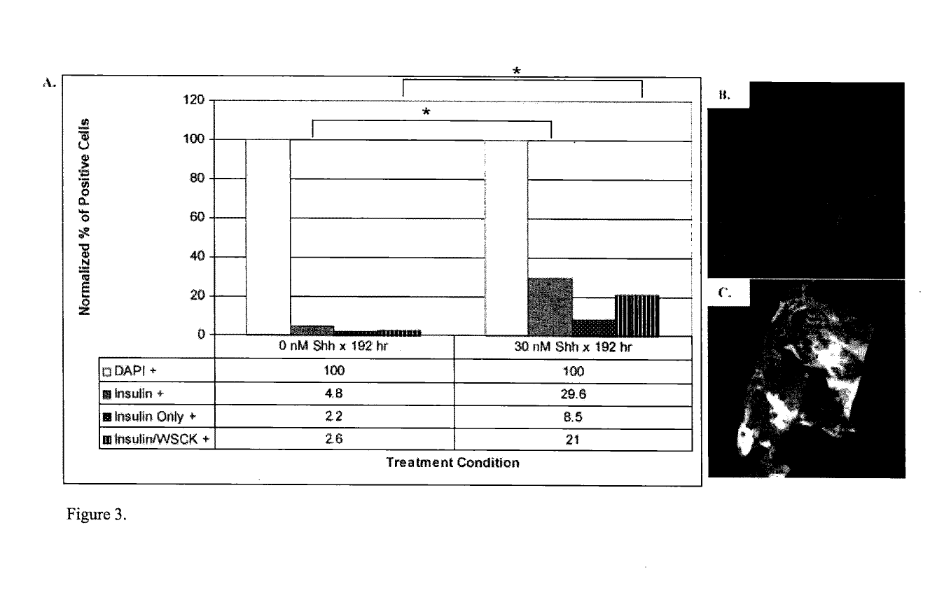

To confirm the in vivo expansion of the insulin-positive compartment, p16^{-/-} ductal cells were exposed to recombinant Shh×192 hrs. Cell counts after immunofluorescent staining for DAPI, WSCK, and insulin demonstrate that p16^{-/-} ductal cells exposed to 30 nM recombinant Shh result in a profound expansion of insulin-positive cells (FIGS. 3B-C): exposure to Shh resulted in a 6-fold increase of such cells from 4.8% to 29.6% (P=0.046). While the cells positive for insulin alone increased from 2.2% to 8.5% (P=0.19), the expansion was most marked for cells positive for both WSCK and insulin (2.6% versus 21.0%, P=0.042; FIG. 3A). It is possible, then, that the mechanism underlying the increased amount of insulin-positive cells involves transdifferentiation from ductal to endocrine phenotype.

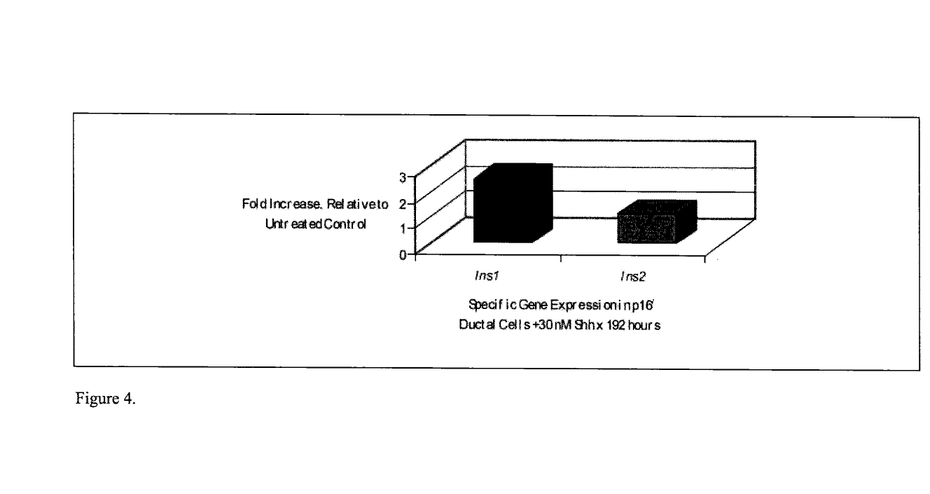

The synergistic effect of Shh and p16^{-/-} to stimulate the transcriptional machinery of murine insulin production is further demonstrated by quantitative real time PCR for murine Ins1 and Ins2 genes (FIG. 4). Expression of Ins1 increased 250% (2.5-fold) after p16^{-/-} ductal cells were treated with recombinant Shh×192 hours, relative to untreated ductal cells. P16−/− ductal cells treated with recombinant Shh×192 hours demonstrated a more modest 15% (1.15-fold) increase in Ins2 expression.

The prevalence of the WSCK- and insulin-positive cells in the Shh-treated p16^{-/-} ductal cells suggest that expansion of insulin producing cells might result, to a degree, from differentiation of the adult, terminally differentiated WSCK-positive epithelium. Whether these differentiated cells also carry progenitor markers or revert to an intermediate de-differentiated state, though, is not clear. Ultimately, though, the synergism of Shh overexpression with the p16 knockout allows for the transformation of terminally differentiated murine ductal epithelial cells to insulin-producing β-cells, demonstrated at both the transcriptional and translational level. While functional assays are ongoing, the ability to transform terminally differentiated epithelial cells into insulin-producing cells, in both the in vivo and in vitro setting, sheds light a possible novel therapeutic modality for the treatment of diabetes.

### Example 3

### Challenging the Role of the p16 Mutation in the Development of Pancreatic Adenocarcinoma

In mice, Shh misexpression in the pancreas results in the formation of mucinous atypical epithelial structures that resemble certain features of PanINs; however, in the model described in this example no cancers were identified. This study suggest that Shh may be sufficient for initiation, but insufficient for progression to cancer. Inactivation of p16 is found in virtually all pancreatic cancers. The role of p16 is thought to involve progression but not initiation since p16−/− mice have normal pancreata. The inventors' goal in this example was to determine the effects of cell cycle inhibition with Shh misexpression.

To develop a model to better delineate the role of Shh with p16/p19 deficiency in pancreatic regeneration, the inventors used a 1^{st} generation PdxShh mice and bred them to a p16/p19−/− knockout mouse. The phenotype that resulted challenged the role of the p16 mutation in the development of pancreatic adenocarcinoma and provided surprising results in terms of pancreatic regeneration.

Misexpression of sonic hedgehog resulted in the formation of structures that retain many features of PanINs. In this model, no cancers have been identified. It is believed that Shh is sufficient for initiation of PanIN lesions, but not for progression to adenocarcinoma. P16 mutations are found in up to 95% of human pancreatic adenocarcinoma specimens. Studies from a genetically engineered kRAS model suggest that a homozygous deficiency of p16 promotes PanIN to adenocarcinoma progression. The aim of the current study was to create a better model with which to understand the role of the Shh initiator and p16^{-/-} progressor in the development of pancreatic adenocarcinoma. However the phenotypic results were unexpected based on the present state of the field.

At one month, none of the animals exhibited neoplasia. However, by three months, the animals that overexpressed sonic developed nuclear atypia and mucinous metaplasia, lesions reminiscent of PanIN formation. The mice that overexpressed sonic with the p16 knockout background did not develop gastrointestinal metaplasia. Alcian Blue and PAS stains demonstrated that animals overexpressing sonic have a persistent and progressive intestinal metaplasia. Animals overexpressing Shh with the p16 knockout demonstrated an attenuated phenotype with minimal amounts of metaplasia.

Wildtype animals have a ductal area comprising less than 1% of total pancreatic area. Animals that misexpress sonic have highly atypical ducts. Quantitatively, their ducts comprise 50% of their total pancreatic area seen here with CK-19 staining for ductal epithelium. In animals that misexpress Shh with the p16 knockout background, there is a 5-fold reduction in ductal area compared animals overexpressing Shh. Here, ductal area comprises 9% of total pancreatic area, trending to that of a control. This phenotypic differences were maintained through 3 months.

Wildtype animals have acinar compartments that comprise 90-95% of their total pancreatic area. In mice overexpressing Shh, the acinar compartment is small and poorly formed, depicted here with a stain for amylase. Quantitatively, the acinar compartment comprises on 23% of total pancreatic area about ⅕ of a wildtype. Mice overexpressing Shh with the p16 knockout exhibit acinar compartments that are larger and well-formed. Quantitatively, their acinar compartments make up 58% of total pancreatic area—over double the size of animals overexpressing Shh alone. Again, normalization in this compartment persists through 3 months.

Using stains for islet markers, the inventors observed that animals overexpressing Shh had islets that were poorly formed 1-2 cells units. The total islet area in these mice was reduced to approximately 10% of a control. However, in mice overexpressing Shh with the p16 knockout, the inventors saw normalization of the endocrine compartment. In fact, the total islet area of these mice was 60% of a wildtype and equivalent to a control. These studies suggest that homozygous deletions of p16 may not necessarily result in pancreatic neoplasia, but might have the ability to reestablish a normal pancreatic phenotype.

Analysis of ki67 failed to demonstrate any significant differences in proliferation mice as a source of the phenotypic changes. Most pancreatic lineages arises from progenitors that originate from and resemble pancreatic epithelium. A plausible hypothesis might be that the combination of Shh misexpression and the p16 knockout allows for the maintenance of a progenitor population.

Under normal conditions, Pdx destines a subset of cells in the embryonic pancreas to an exocrine fate. In the adult, Pdx expression can be detected in the adult islet but not in the ducts. However, in this mouse that overexpress sonic hedgehog with a p16 knockout, there is Pdx activity in the ducts, recapitulating pancreatic embryonic development.

p48 is expressed in the embryonic pancreas in cells that are destined to an acinar fate, seen by the inventors in an embryonic day 15.5 pancreas. It is not detectable in the adult mouse pancreatic duct. However, in this mouse overexpressing Shh with the p16 knockout, p48 is expressed not only in the acinar, but also the ductal compartment.

ngn3 is found in the embryonic pancreas in cells destined to an endocrine fate, seen by the inventors in an embryonic 15.5 day pancreas. ngn3 is not detectable in the adult pancreas. In these adult mice that overexpress Shh with the p16 knockout, ngn3 activity is found in the ductal epithelium, which is reminiscent of embryonic development. This work suggests that the overexpression of sonic hedgehog may facilite the maintenance of progenitor cells or a pancreatic progenitor epithelium.

Combining Shh overexpression and p16 knockout results in the normalization of pancreatic phenotype. It remains possible that under certain circumstances the p16 knockout does act as a progressor. However, in combination with Shh overexpression, the p16 knockout facilitates normalization and appears to challenge the role of the p16 mutation in the development of pancreatic cancer. The mechanisms by which these findings occurs is still unknown, but might involved maintenance of a progenitor populations.

These findings suggest that the additive effect of Shh overexpression and a homozygous p16 deletion induces a normalization of a pancreatic neoplastic phenotype. These findings challenge the currently accepted role of the p16 mutation as a progressor of pancreatic adenocarcinoma. The mechanism indicates an expansion of a progenitor cell population. As well, it can be appreciated that the combination of overexpression of Shh and loss of p16 allows for the maintenance of a primordial epithelium, which is capable of repopulating all three compartments of the pancreas, including the islets, ducts, and acinar cells. There is also observed an expansion of ngn3 progenitor cells and restoration of islet cell mass, this difference principally noted in the beta cell compartment.

### Example 4

### Paradoxical Inhibition of Ductal Proliferation Due to Shh Misexpression with P16^{-/-} is Accompanied by Pluripotential Cell Expansion

Shh misexpression in the murine pancreas results in mucinous atypical epithelial structures that resemble features of PanINs; no cancers were identified. Early studies suggest Shh may be sufficient for initiation but insufficient for progression to cancer. Inactivation of p16 is found in virtually all pancreatic cancers. Its role may involve progression but not initiation as p16^{-/-} mice have normal pancreata. The inventors goal in this example was to determine if Shh misexpression with p16^{-/-} was sufficient to induce adenocarcinoma and if not, to identify mechanisms that induce normalization.

Bigenic mice were created that misexpress Shh and contain a homozygous deletion of p16. The phenotypes of PdxShh, PdxShh;p16^{-/-} and their age-matched controls were histologically evaluated at 1 and 3 months.

Wildtypes and p16^{-/-} controls have normal pancreata at all timepoints. Through 1 mo, PdxShh;p16^{-/-} mice resemble PdxShh counterparts with poorly formed islets, a small peripheral rim of acinar tissue and extensive central ductal replacement. There is no identifiable neoplasia. Phenotypic separation is identified by 3 mo. The pancreata of PdxShh mice develop progressive mucinous metaplasia and atypia of the epithelial compartment that resembles features of PanINs. The acinar and endocrine compartments remain small and poorly formed. However, PdxShh;p16^{-/-} mice exhibit normal ducts with a low cuboidal epithelium and without metaplasia and atypia. The areas of the acinar and endocrine compartments are similar to controls. This normalization of phenotype is associated with a decrease in ductal proliferation, a decrease in pRB expression, and alterations in cyclin D species expression. There is also an increase in progenitor marker expression (e.g., Pdx1, ngn3, p48, nestin) in bigenic animals.

Loss of p16 with Shh misexpression does not facilitate pancreatic adenocarcinoma development. Rather, the combination normalizes phenotype, inhibits ductal proliferation with associated pRB and cyclin D alterations, and preserves a pluripotential cell population, reminiscent of the embryonic state and capable of postnatal development.

### Example 5

### P16−/− Adult Ductal Cells, Exposed to Shh, Secreted Insulin

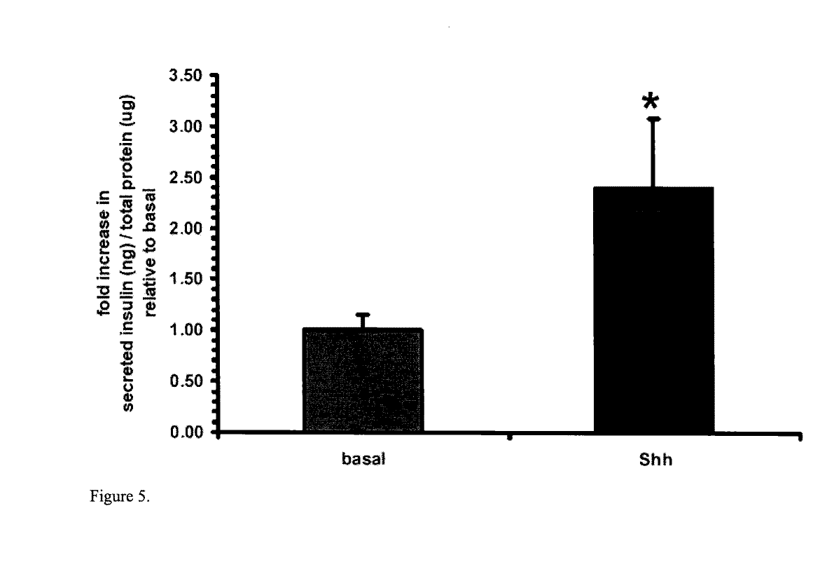

P16−/− adult ductal cells, exposed to Shh, demonstrate a significant increase in secreted insulin. P16−/− adult ductal cells were exposed to 0 or 30 nM recombinant Shh for a predetermined duration of time. Buffer, with secreted insulin, was collected, and the total cellular protein was extracted. Concentrations of secreted insulin and total extracted cellular protein were determined. Secreted insulin concentrations were normalized to their respective total cellular protein concentrations. Data in FIG. 5 is plotted as fold increase in secreted insulin (ng)/total protein (ug) relative to basal, untreated p16−/− duct cells. P16−/− adult ductal cells, treated with 30 nM recombinant Shh, demonstrated a significant, 2.4 fold increase in secreted insulin, relative to basal insulin secretion in untreated cells (P=0.039).

TABLE 2

| | fold increase in secreted insulin (ng)/ | |
|---|---|---|
| cell line | total protein (ug) relative to basal | SEM |
| p16 −/− | 1.00 | 0.15 |
| duct basal | | |
| Shh | 2.40 | 0.68 |

It should be noted that the above description, attached figures and their descriptions are intended to be illustrative and not limiting of this invention. Many themes and variations of this invention will be suggested to one skilled in this and, in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments. Accordingly, the exemplary embodiments according to this invention, as set forth above, are intended to be illustrative, not limiting. All publications, patents and patent applications cited herein are hereby incorporated by reference in their entirety for all purposes.

### REFERENCES

¹S. Bonner-Weir, Journal of molecular endocrinology 24 (3), 297 (2000).
²Y. Dor, J. Brown, O. I. Martinez et al., Nature 429 (6987), 41 (2004).
³O. Strobel, Y. Dor, A. Stirman et al., Proceedings of the National Academy of Sciences of the United States of America 104 (11), 4419 (2007).
⁴A. Sharma, D. H. Zangen, P. Reitz et al., Diabetes 48 (3), 507 (1999).
⁵J. F. Habener, D. M. Kemp, and M. K. Thomas, Endocrinology 146 (3), 1025 (2005).
⁶V. M. Schwitzgebel, Molecular and cellular endocrinology 185 (1-2), 99 (2001).
⁷D. A. Stoffers, Thomas, M. K., Habener, J. F., Trends in Endocrinology and Metabolism 8 (4), 145 (1997).
⁸G. Gu, J. Dubauskaite, and D. A. Melton, Development (Cambridge, England) 129 (10), 2447 (2002).
⁹V. M. Schwitzgebel, D. W. Scheel, J. R. Conners et al., Development (Cambridge, England) 127 (16), 3533 (2000).
¹⁰X. Xu, J. D'Hoker, G. Stange et al., Cell 132 (2), 197 (2008).
¹¹G. Gradwohl, A. Dierich, M. LeMeur et al., Proceedings of the National Academy of Sciences of the United States of America 97 (4), 1607 (2000).
¹²G. Teitelman and J. K. Lee, Developmental biology 121 (2), 454 (1987).
¹³S. Alpert, D. Hanahan, and G. Teitelman, Cell 53 (2), 295 (1988).
¹⁴R. W. Dudek, I. E. Lawrence, Jr., R. S. Hill et al., Diabetes 40 (8), 1041 (1991).
¹⁵J. M. Slack, Development (Cambridge, England) 121 (6), 1569 (1995).
¹⁶S. Bonner-Weir, M. Taneja, G. C. Weir et al., Proceedings of the National Academy of Sciences of the United States of America 97 (14), 7999 (2000).
¹⁷Y. Choi, M. Ta, F. Atouf et al., Stem cells (Dayton, Ohio) 22 (6), 1070 (2004).
¹⁸M. K. Thomas, N. Rastalsky, J. H. Lee et al., Diabetes 49 (12), 2039 (2000).
¹⁹S. Ahn and A. L. Joyner, Nature 437 (7060), 894 (2005).
²⁰V. Levy, C. Lindon, B. D. Harfe et al., Developmental cell 9 (6), 855 (2005).
²¹C. D. Peacock, Q. Wang, G. S. Gesell et al., Proceedings of the National Academy of Sciences of the United States of America 104 (10), 4048 (2007).
²²S. Liu, G. Dontu, I. D. Mantle et al., Cancer research 66 (12), 6063 (2006).
²³C. Li, D. G. Heidt, P. Dalerba et al., Cancer research 67 (3), 1030 (2007).
²⁴M. Hebrok, S. K. Kim, B. St Jacques et al., Development (Cambridge, England) 127 (22), 4905 (2000).
²⁵M. K. Thomas, O. N. Devon, J. H. Lee et al., The Journal of clinical investigation 108 (2), 319 (2001).
²⁶J. Krishnamurthy, M. R. Ramsey, K. L. Ligon et al., Nature 443 (7110), 453 (2006).
²⁷V. Janzen, R. Forkert, H. E. Fleming et al., Nature 443 (7110), 421 (2006).
²⁸S. P. Thayer, M. P. di Magliano, P. W. Heiser et al., Nature 425 (6960), 851 (2003).
²⁹N. E. Sharpless, N. Bardeesy, K. H. Lee et al., Nature 413 (6851), 86 (2001).
³⁰F. S. Schreiber, T. B. Deramaudt, T. B. Brunner et al., Gastroenterology 127 (1), 250 (2004).

What is claimed is:

1. A method of inducing β-cell neogenesis from epithelial cells, comprising exposing epithelial cells that have a disrupted G1-S cell cycle transition to a hedgehog protein in an amount effective to stimulate β-cell neogenesis from said epithelial cells.

2. The method according to claim 1, wherein said isolated hedgehog protein is desert hedgehog, indian hedgehog, or sonic hedgehog.

3. The method according to claim 1, wherein said method is carried out in vitro.

4. The method according to claim 1, wherein said method is carried out in vivo.

5. The method according to claim 1, wherein said epithelial cells that have a disrupted G1-S cell cycle transition contain a dysfunctional INK4a/ARF locus.

6. The method according to claim 1, wherein said epithelial cells that have a disrupted G1-S cell cycle transition contain one or more dysfunctional p53, Rb, cyclin, cyclin-dependent kinase, or cyclin-dependent kinase inhibitor.

7. A method of inducing pancreatic epithelium to form a primordial epithelium capable of regenerating pancreatic islet, duct and acinar cells, comprising: (a) disrupting the G1-S cell cycle transition in the pancreatic epithelium; and (b) exposing the resultant epithelium to a hedgehog protein in an amount effective to induce said pancreatic epithelium to form a primordial epithelium capable of regenerating pancreatic islet, duct and acinar cells from said pancreatic epithelium.

8. The method according to claim 7, wherein said isolated hedgehog protein is desert hedgehog, indian hedgehog, or sonic hedgehog.

9. The method according to claim 7, wherein said method is carried out in vitro.

10. The method according to claim 7, wherein said method is carried out in vivo.

11. The method according to claim 7, wherein said epithelial cells that have a disrupted G1-S cell cycle transition contain a dysfunctional INK4a/ARF locus.

12. The method according to claim 7, wherein said epithelial cells that have a disrupted G1-S cell cycle transition contain one or more dysfunctional p53, Rb, cyclin, cyclin-dependent kinase, or cyclin-dependent kinase inhibitor.

13. A method of treating a deficiency of insulin in a patient, comprising inducing β-cell neogenesis in pancreatic epithelial cells of said patient by: (a) disrupting G1-S cell cycle transition in said pancreatic epithelial cells of the patient; and (b) exposing said pancreatic epithelial cells to a hedgehog protein in an amount effective to stimulate β-cell neogenesis from said pancreatic epithelial cells wherein the level of insulin is raised in said patient containing the pancreatic epithelial cells having undergone β-cell neogenesis.

14. The method according to claim 13, wherein said isolated hedgehog protein is desert hedgehog, indian hedgehog, or sonic hedgehog.

15. The method according to claim 13, wherein said epithelial cells that have a disrupted G1-S cell cycle transition contain a dysfunctional INK4a/ARF locus.

16. The method according to claim 13, wherein step (a) of disrupting the G1-S cell cycle transition of said epithelial cells includes rendering dysfunctional p53, Rb, a cyclin, a cyclin-dependent kinase, a cyclin-dependent kinase inhibitor, or a combination thereof.

17. The method according to claim 13, wherein said patient is afflicted with diabetes.
